(19) United States
(12) Patent Application Publication
(10) Pub. No.: US 2011/0118347 A1
(43) Pub. Date: May 19, 2011

(54) TREATMENT Of RINGWORM

(75) Inventor: Michael William Fowler, Derbyshire (GB)

(73) Assignee: Vernolix Limited, London (GB)

(21) Appl. No.: 12/995,283

(22) PCT Filed: May 29, 2009
(86) PCT No.: PCT/GB09/50595

§ 371 Date: Jan. 6, 2011

(30) Foreign Application Priority Data
May 30, 2008 (GB) 0809816.2

(51) Int. Cl. A61K 31/336; C07D 303/38; C07C 59/42; A61K 31/201; C07D 303/46; C07C 235/28; A61K 31/16; C07C 59/44; C07C 69/732; A61K 31/231; C07C 59/01; C07C 59/105; A61K 31/20; C07C 235/04; A61P 31/10

(52) U.S. Cl. 514/475; 549/561; 554/219; 514/560; 549/553; 554/61; 514/627; 514/549; 514/558; 514/625

(57) ABSTRACT

A compound of the formula CH₃(CH₂)ₙ—R^{A}—(CH₂)m-(CH═CH)_{q}—(CH₂)ₚ—X wherein R^{A}=(a); q=0 or 1; m and p are independently selected integers of at least 1; n=0 or an integer of at least 1; X is a polar group selected from —CO₂R^{S}, where R^{s}═H, Na, K, —NHR^{I}, —NR^{I}R^{II} or R^{III}; —CH₃OH; —CH₃NH₂; —CH₃NHR^{I}; or —CH₃NR^{I}R^{II}; wherein R^{I}, R^{II} and R^{III} are independently selected from C₁-C₆ alkyl or an aryl group, preferably a hydrophilically substituted C₁-C₆ alkyl or aryl group, or R^{III} is an esterified glycol; and wherein 20>m+n+p+2q≧2; is useful for the treatment of ringworm infection caused by Trichophyton tonsurans.

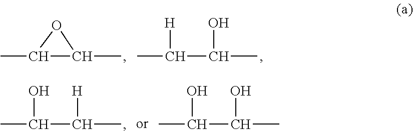

The present invention relates to the use of fatty acid epoxide, diol or alcohol derivatives in the preparation of compositions for the treatment of ringworm and to a method of treatment of ringworm with compositions comprising a fatty acid epoxide, diol or alcohol derivative. The present invention is particularly directed to the treatment of ringworm infection caused by Trichophyton tonsurans in the skin of humans and other mammals, including Equidae such as horses.

Ringworm is a general term that encompasses skin infections caused by various dermatophite fungi. The Latinate clinical description for a specific ringworm infection includes the group “Tinea” followed by the affected part of the body. For example, Tinea capitis refers to an infection of the hair or scalp, Tinea corporis refers to an infection of the body, Tinea cruris refers to an infection of the groin, perineum or perianal region, Tinea pedis refers to an infection of the feet (which is why it is commonly known as athlete's foot), Tinea unguium refers to an infection of the nails, Tinea barbae refers to an infection of the beard region and Tinea manuum refers to an infection of the hand.

The dermatophyte fungi which cause ringworm come from three genera: Trichphyton, Microsporon and Epidermophyton, but the particular pathogen responsible for a particular type of skin infection varies with the part of the body afflicted. There are three main pathogens which are considered to invade the epidermis of the skin: T. rubrum, M. canis and E. floccosum, but this list is not exhaustive. T. tonsurans is another ringworm afflicting pathogenic fungus but it is believed that this invades the dermis of the skin.

A dermatophyte fungal infection often reveals itself as a red or silvery patch and is itchy. Scalp and beard infections may cause patches of baldness. In scalp infections, the lymph nodes at the back of the neck may become swollen and tender.

T. tonsurans is known to cause classical ringworm symptoms, wherein the patch is round or ring-shaped and the edge is red and may be raised. The patch spreads outwards and the centre may heal and go back to a normal skin colour. There may be several patches, not just one, and they may blister and ooze pus. Patches can range from a few millimetres to a few centimetres in size. It is to the treatment of classical ringworm infections caused by T. tonsurans which the present invention is directed.

Classical ringworm infections are often treated using antifungal powders, lotions and creams applied topically. The active principle in such compositions includes pharmaceutical compounds such as clotrimazole and miconazole, imidazoles (e.g. ketoconazole) or terbinafine.

Tinea versicolor or Pityriasis versicolor is a common skin infection caused by Malassezia yeasts, such as Malassezia furfur (formerly termed Pityrosporum ovale) and Malassezia globosa. Though symptoms of these yeast infections may be similar in appearance and may be commonly referred to as ringworm, they are not true ringworm infections and are not referred to medically as such. For the purposes of the present application, yeast infections are not included within the term ringworm.

The triglycerides of fatty acids having in chain alkenyl unsaturation and hydroxy or epoxy groups, such as the triglyceride of 12:13-epoxyoctadec-9-enoic acid can be found in nature. Plants of the genus Vernonia, such as Vernonia scorpioides, Vernonia galamensis and Vernonia globosa are particularly rich sources of these triglycerides and oils extracted from these plants are known for the treatment of skin complaints. For example, GB 2 353 706 discloses the use of Vernonia anthelmintica (c.f. Centratherum antheminticum) extracts for the treatment of fungal infections of the skin and nails. Similarly, WO 2007/077452 discloses the use of Vernonia galamensis extracts in the treatment of lesions of the skin which may be caused by fungal infection.

WO 2007/113851 discloses a composition for hair loss prevention and/or hair growth promotion comprising an extract obtained from the plant Vernonia sp.

EP1917855A1 discloses the use of unsaturated fatty acid salts of e.g. linoleic acid, linolenic acid, myristoleic acid and palmitoleic acid as a surfactant in a skin disinfectant. The disinfectant is disclosed to be a useful remedy for a ringworm infectious disease, and exemplifies Trichophyton rubrum and Trichophyton mentagrophytes. Trichophyton rubrum and Trichophyton mentagrophytes are known to invade the epidermis of the skin.

WO2003/25213 discloses a dermatological composition useful in the treatment of a variety of skin, muscle and joint disorders including ringworm. The composition comprises nitrous oxide and at least one essential fatty acid or ester selected from linoleic acid, arachidonic acid, linolenic acid, gamma-linolenic acid and the lower alkyl esters thereof.

US2004/0091506 discloses a topical antifungal preparation comprising triacetin and tolnaftate. It also discloses that tolnaftate is used topically for the treatment of certain dermatophytoses cases by Trichophyton rubrum, Trichophyton tonsurans, Microsporum canis, Microsporum audouini or Epidermophyton floccosum. US2007/0098805 discloses compositions comprising griseofulvin for the treatment of a fungal infection of, amongst others, Trichophyton tonsurans, and reference is also made to treatment of ringworm. US2005/0107344 discloses that the fungal infections tinea capitas and ringworm (Trichophyton tonsurans) may be treated with compositions containing certain protonated phosphate compounds.

There is a need for effective medicaments for the treatment of ringworm, particularly ringworm caused by Trichophyton tonsurans.

An object of the present invention is to provide an active principle for use in the preparation of medicaments for the treatment of ringworm, particularly ringworm caused by Trichophyton tonsurans.

The present invention, in its various aspects, is as set out in the accompanying claims.

In a first aspect, the present invention provides the use of a compound of Formula I:

CH₃(CH₂)ₙ—R^{A}—(CH₂)ₘ—(CH═CH)_{q}—(CH₂)ₚ—X Formula I

wherein

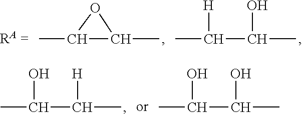

q=0 or 1;
m and p are independently selected integers of at least 1;
n=0 or an integer of at least 1;
X is a polar group selected from —CO₂R^{S}, Where R^{S}═H, Na, K, —NHR¹, —NR^{I}R^{II} or R^{III}; —CH₂OH; —CH₂OH; —CH₂NH₂; —CH₂NHR^{I}; or —CH₂NR^{I}R^{II};
wherein R^{I}, R^{II} and R^{III} are independently selected from C₁-C₆ alkyl or an aryl group, preferably a hydrophilically substituted C₁-C₆ alkyl or aryl group, or R^{III} is an esterified glycol; and
wherein 20>m+n+p+2q≧2;
in the preparation of a composition for the treatment of ringworm infection caused by Trichophyton tonsurans.

In a second aspect, the present invention provides a method for the treatment of ringworm infection caused by Trichophyton tonsurans comprising contacting a ringworm infected portion of skin with a composition comprising a compound of Formula I:

CH₃(CH₂)ₙ—R^{A}—(CH₂)ₘ—(CH═CH)_{q}—(CH₂)ₚ—X Formula I

wherein

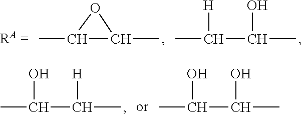

q=0 or 1;
m and p are independently selected integers of at least 1;
n=0 or an integer of at least 1;
X is a polar group selected from —CO₂R^{S}, where R^{S}═H, Na, K, —NHR¹, —NR^{I}R^{II} or R^{III}; —CH₂OH; —CH₂OH; —CH₂NH₂; —CH₂NHR^{I}; or —CH₂NR^{I}R^{II};
wherein R^{I}, R^{II} and R^{III} are independently selected from C₁-C₆ alkyl or an aryl group, preferably a hydrophilically substituted C₁-C₆ alkyl or aryl group, or R^{III} is an esterified glycol; and
wherein 20>m+n+p+2q≧2.

The mode of activity of the relevant compounds for treatment of T. tonsurans ringworm is not understood but, whatever the mode may be, that such compounds can demonstrate activity against infections seated in the skin at levels deeper than the epidermis is surprising.

In the present invention,

R^{A} is preferably

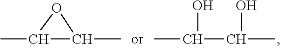

more preferably

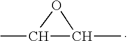

In the present invention, q is preferably =1. The compounds useful in the present invention which comprise ethylenic unsaturation can provide that unsaturation in cis or trans forms. The cis form is preferred as this is believed to sterically hinder the epoxy or hydroxy groups, such as may prevent self association of the compounds, for example, in polymerization. Accordingly, when q=1 then —CH═CH— is preferably cis.

In the present invention, R^{S} is preferably ═H, Na, K, —NHR^{I}, —NR^{I}R^{II}, more preferably R^{S}═H, Na or K, most preferably R^{S}═H.

In the present invention, it is preferred that:

n=0 to 6;
m=1 to 3; and
p=5 to 9.

Preferably 17≧m+n+p+2q≧4, more preferably m+n+p+2q=14.

In the following embodiments, the above preferments may apply.

In one embodiment the compound of Formula I is a compound of Formula II:

CH₃(CH₂)ₙ—R^{A}—(CH₂)ₘ—(CH═CH)_{q}—(CH₂)ₚ—CO₂R^{S} Formula II

wherein R^{S}═H, Na, K, —NHR^{I}, —NR^{I}R^{II} or R^{III}.

In another embodiment the compound of Formula I is of Formula III:

CH₃(CH₂)ₙ—R^{A}—(CH₂)ₘ—CH═CH—(CH₂)ₚ—CO₂R^{S} Formula III

wherein
n=0 to 6
m=1 to 3
p=5 to 9
and wherein 17≧m+n+p+2q≧4, preferably m+n+p+2q=14.

Specific preferred compounds of Formula I are the octadecenoic acid derivatives:

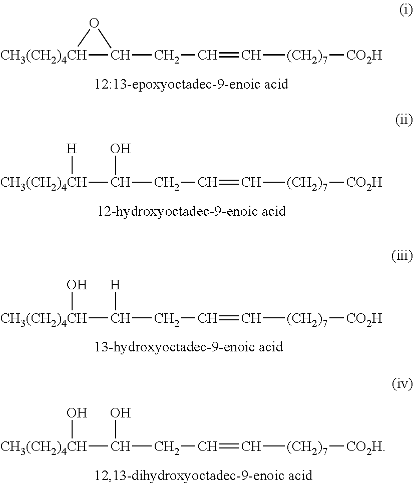

Further specific preferred compounds of Formula I are octadecenoic acid amide derivatives in the form of

primary amides of structure:

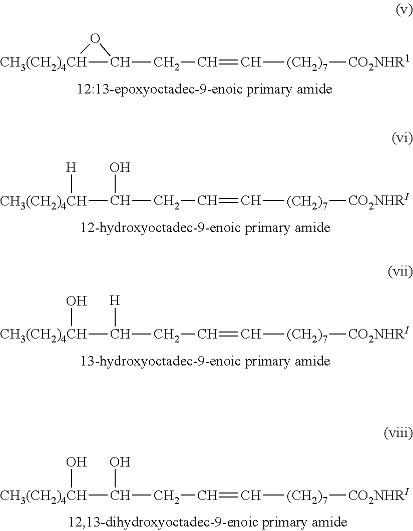

or octadecenoic acid amide derivatives in the form of secondary amides of structure:

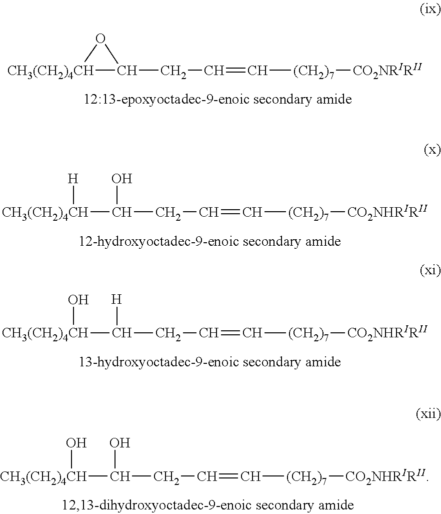

Further specific preferred compounds of Formula I are octadecenoic acid esters of structure:

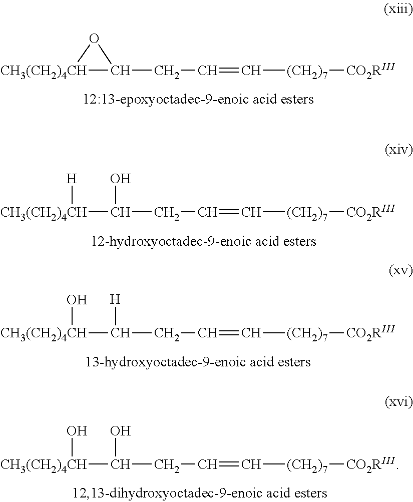

In yet another embodiment the compound of Formula I is an alkanoic acid of Formula IV:

CH₃(CH₂)ₙ—R^{A}—(CH₂)ₘ₊ₚ₊₂—CO₂R^{S} Formula IV

wherein

n=0 to 6

m+p+2=4 to 18

and wherein 19≧m+n>3.

Specific compounds of Formula IV are the octadecanoic acid derivatives:

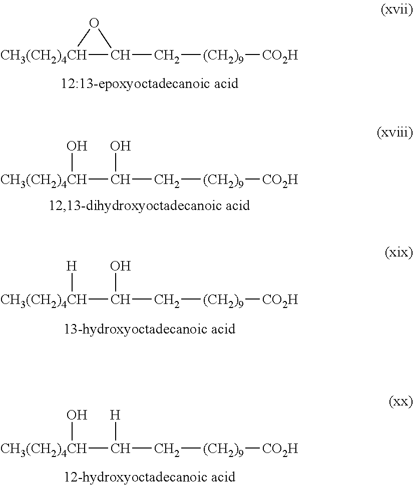

Further specific compounds of Formula IV are octadecanoic acid amide derivatives in the form of:

primary amides of structure:

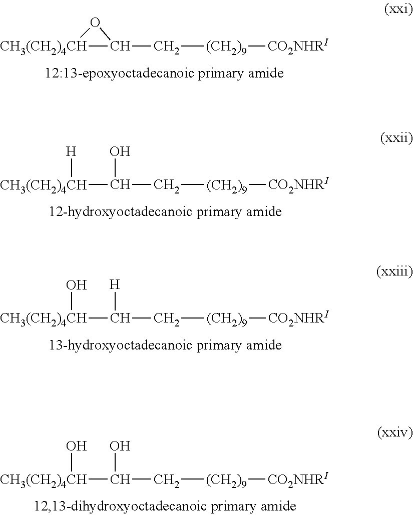

or octadecenoic acid amide derivatives in the form of secondary amides of structure:

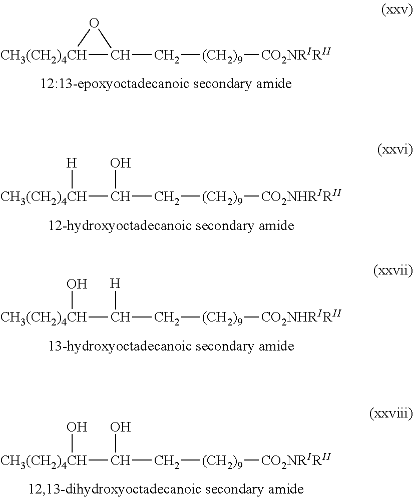

Further specific preferred compounds of Formula IV are octadecanoic acid esters of structure:

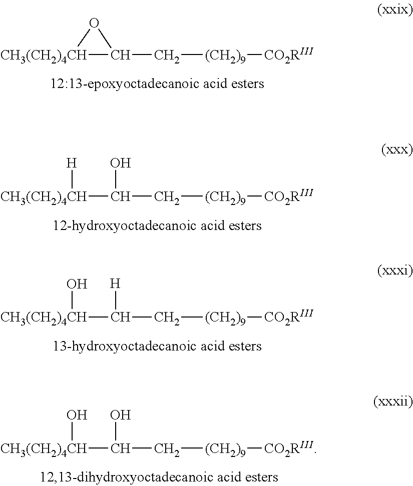

The composition for the treatment of ringworm can comprise one or more of the active compounds hereinbefore described. The composition may also comprise one or more other conventional components, such as an appropriate carrier medium, thereby to provide the active compound in a format which preferably combines regulated release coupled with maintaining a consistent therapeutic level of the active compound at the prime fungal infection site. The carrier medium may, for example, be chosen so as to provide the composition in the form of a cream, lotion, gel or ointment. The carrier medium is preferably a lotion to aid penetration and availability of the active compound at the fungal infection site.

In one embodiment of the present invention, the composition may be a medicament.

In another embodiment, for example where either prevention of or avoidance of a recurrence of a ringworm infection is desired, the composition may be a cosmetic, a body lotion, a shampoo or a body wash.

It will be appreciated by those skilled in the art that compounds of Formula I should be used in an amount sufficient to be effective against the ringworm without any toxicological ramifications for the patient. Depending upon the nature of the formulation e.g. lotion, cosmetic, ointment, shampoo, body wash or injectable preparation, its nature of administration e.g. rub/roll/spray-on/patch (lotion, cosmetic, ointment), wash-on/off (shampoo, body wash) or injection (injectable preparation), and the particular active ingredient of Formula I employed in the formulation, the formulation will typically comprise the active ingredient of Formula I in an amount of from 0.001 to 10% by wt, preferably 0.001 to 5% by wt, more preferably 0.001 to 1% by wt, based on the total weight of the composition.

Irrespective of the nature of the treatment formulation, the active ingredient should be present in the treatment formulation in an amount so as to provide preferably at least 1 ppm a.i. of Formula I at the locus of ringworm infection, more preferably at least 100 ppm a.i. of Formula I at the locus of infection.

The present inventions shall now be described further with reference to the following examples.

a) Effect of Compounds of Formula I Against T. tonsurans

Compound (i) (12:13-epoxyoctadec-9-enoic acid) and Compound (iv) (12,13-dihydroxyoctadec-9-enoic acid) were each screened for activity against fungal lawns of T. tonsurans. Both compounds were found to have effective fungicidal activity.

b) Penetration of Compounds of Formula I into Mammalian Skin

Formulations for topical administration may be prepared by the incorporation of compounds (i) and (iv) into, for example, a standard pharmaceutical grade lanolin-based cream in an amount of 5% by wt, based on the total weight of the formulation. The formulation may be applied to the outer surface of a section of living pig skin. After one hour, the skin can be tested to determine the penetration of the active compounds in to the dermis and epidermis. The results of such tests will reveal that a significant amount of compounds (i) and (iv) has penetrated the dermis, rather than being held entirely on the skin surface or in the upper parts of the epidermis.

From the above and the fact that T. tonsurans is found in the dermis an optimised treatment can be determined.

1. Use of a compound of Formula I:
CH₃(CH₂)ₙ—R^{A}—(CH₂)ₘ—(CH═CH)_{q}—(CH₂)ₚ—X Formula I
wherein 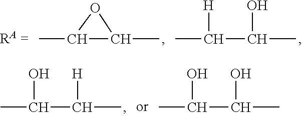
q=0 or 1;
m and p are independently selected integers of at least 1;
n=0 or an integer of at least 1;
X is a polar group selected from —CO₂R^{S}, where R^{S}═H, Na, K, —NHR^{I}, —NR^{I}R^{II} or R^{III}; —CH₂OH; —CH₂NH₂; —CH₂NHR^{I}; or —CH₂NR^{I}R^{II};
wherein R^{I}, R^{II} and R^{III} are independently selected from C₁-C₆ alkyl or an aryl group, or R^{III} is an esterified glycol;
and
wherein 20>m+n+p+2q≧2;
in the preparation of a composition for the treatment of ringworm infection caused by Trichophyton tonsurans.

2. Use of a compound as claimed in claim 1, wherein Formula I is a compound of Formula II
CH₃(CH₂)ₙ—R^{A}—(CH₂)ₘ—(CH═CH)_{q}—(CH₂)ₚ—CO₂R^{S} Formula II
wherein R^{S}═H, Na, K, —NHR^{I}, —NR^{I}R^{II} or R^{III}.

3. The use of claim 1, wherein R^{S}═H, Na or K.

4. The use of claim, wherein R^{S} is an esterified glycol.

5. The use of claim 1, wherein 17>m+n+p+2q≧4.

6. The use of claim 5, wherein m+n+p+2q=14.

7. The use of claim 1, wherein q=1.

8. The use of claim 1, wherein q=0.

9. The use of claim 2, wherein the compound of Formula II is one or more of

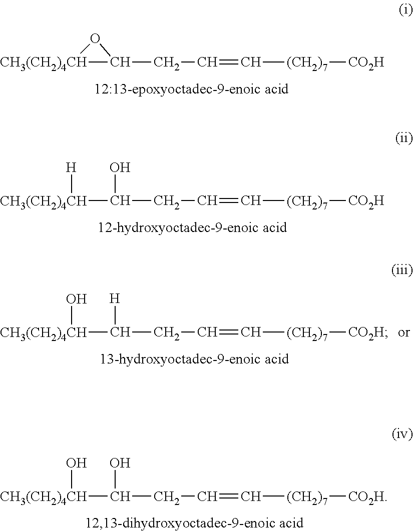

10. The use of claim 9 wherein the compound of Formula II is 12:13-epoxyoctadec-9-enoic acid.

11. Method for the treatment of ringworm inflection caused by Trichophyton tonsurans comprising contacting a ringworm infected portion of skin with a composition comprising a compound of Formula I:
CH₃(CH₂)ₙ—R^{A}—(CH₂)ₘ—(CH═CH)_{q}—(CH₂)ₚ—X Formula I
wherein 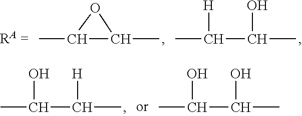
q=0 or 1;
m and p are independently selected integers of at least 1;
n=0 or an integer of at least 1;
X is a polar group selected from —CO₂R^{S}, where R^{S}═H, Na, K, —NHR^{I}, —NR^{I}R^{II} or R^{III}; —CH₂OH; —CH₂NH₂; —CH₂NHR^{I}; or —CH₂NR^{I}R^{II};
wherein R^{I}, R^{II} and R^{III} are independently selected from C₁-C₆ alkyl or an aryl group, or R^{III} is an esterified glycol;
and
wherein 20>m+n+p+2q≧2.

12. Method of claim 11, wherein Formula I is a compound of Formula II
CH₃(CH₂)ₙ—R^{A}—(CH₂)ₘ—(CH═CH)_{q}—(CH₂)ₚ—CO₂R^{S} Formula II
wherein R^{S}═H, Na, K, —NHR^{I}, —NR^{I}R^{II} or R^{III}.

13. Method of claim 11, wherein R^{S}═H, Na or K.

14. Method of claim 11, wherein R^{S} is an esterified glycol.

15. Method of claim 11, wherein 17≧m+n+p+2q≧4.

16. Method of claim 11, wherein m+n+p+2q=14.

17. Method of claim 11, wherein q=1.

18. Method of claim 11, wherein q=0.

19. Method of claim 11, wherein the compound of Formula II is one or more of

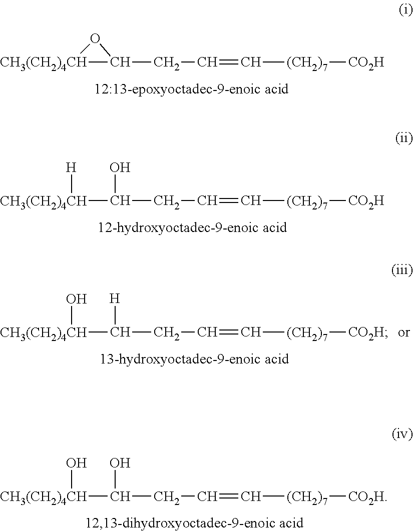

20. Method of claim 11, wherein the compound of Formula II is 12:13-epoxyoctadec-9-enoic acid.
